(19) United States
(12) Patent Application Publication
(10) Pub. No.: US 2021/0100738 A1
(43) Pub. Date: Apr. 8, 2021

(54) OXYGEN DELIVERY BEVERAGE

(71) Applicant: RESPIROGEN, INC., Boulder, CO (US)

(72) Inventors: Robert M. Scribner, Boulder, CO (US); Paul MOUNTFORD, Boulder, CO (US); Mark BORDEN, Boulder, CO (US); Robert T. Scribner, Boulder, CO (US)

(21) Appl. No.: 17/069,394

(22) Filed: Oct. 13, 2020

Related U.S. Application Data
(63) Continuation of application No. PCT/US19/27553, filed on Apr. 15, 2019.
(60) Provisional application No. 62/657,713, filed on Apr. 13, 2018.

(51) Int. Cl. A61K 9/00; A61K 33/00; A61K 31/05; A61K 31/352; A61P 7/00

(52) U.S. Cl. CPC A61K 9/0029; A61K 9/0095; A61P 7/00; A61K 31/05; A61K 31/352; A61K 33/00

(57) ABSTRACT

Disclosed are improved devices, systems and methods of delivering oxygen and/or other therapeutic substances to a living organism, such as a mammal and/or human patient, by delivering and/or circulating microbubble carriers within existing anatomical passages of the organism that are accessible via external body orifices, such as portions of the organism's digestive and/or excretory tracts.

### CROSS-REFERENCE TO RELATED APPLICATIONS

This application in a continuation application of PCT Patent Application Ser. No. PCT/US19/27553 entitled “Oxygen Delivery Beverage,” filed Apr. 15, 2019, which in turn claims the benefit of U.S. Provisional Patent Application Ser. No. 62/657,713 entitled “Oxygen Delivery Beverage,” filed Apr. 13, 2018. The disclosure of each of these applications is incorporated by reference herein in their entireties.

### TECHNICAL FIELD

The invention relates to improved devices, systems and methods of delivering oxygen and/or other therapeutic substances to a living organism, such as a mammal and/or human patient, by delivering and/or circulating substances within existing anatomical passages of the organism that are accessible via external body orifices, such as portions of the organism's digestive and/or excretory tracts. More specifically, disclosed are compounds that utilize microbubble carriers to desirably enable and/or facilitate the transport of substances across tissue membranes into and/or through an organism's cells for satisfactory treatment effects, including use of an oxygen microbubble carrier that mimics the mechanical and gas transport properties of the alveolus to deliver an oxygen payload to tissues (and/or for uptake to the bloodstream) via a patient's digestive tract.

### BACKGROUND OF THE INVENTION

Oxygen is one of the basic essentials for sustaining life. Today's medical technology can supply oxygen to patients experiencing pulmonary failure, otherwise known as respiratory failure, which occurs when the lungs experience significant damage and are unable to supply the body and brain with oxygen. Pulmonary failure may be caused by a variety of conditions including, for example, lung cancer, physical trauma, acute respiratory distress syndrome (ARDS), aerosolized bioterrorism agents, and diseases such as severe acute respiratory syndrome (SARS), pneumonia, tuberculosis, sepsis, and other bacterial or viral infections, physical trauma, and chemical or smoke inhalation. Currently, oxygen can be supplied to patients experiencing pulmonary failure through mechanical ventilation (MV) or extracorporeal membrane oxygenation (ECMO). However, the delivery of oxygen using such methods requires an extensive amount of specialized equipment, and the mortality rate of patients receiving oxygen through MV or ECMO remains high.

Mechanical ventilation, or positive pressure ventilation, uses a ventilator to push air into the lungs through an endotracheal tube or tracheostomy tube. Noninvasive ventilation can be delivered through a face mask for some patients who retain control of their airway (intact gag reflex). For intubated patients, the machine pushes in a mixture of oxygen and other gases until a signal causes the ventilator to stop and allows passive expiration. The ventilator can replace or support spontaneous breathing. The ventilator can be set to coincide with the patient's own breath (triggered) or set to deliver a targeted flow rate or volume of air. The tidal volume is the amount of air delivered with each breath. Low tidal volume ventilation (≤6 mL/kg/predicted body weight) is associated with better outcomes for patients with ARDS. The low tidal volume typically requires a higher respiratory rate (^{˜}35 breaths/min) in order to support adequate tissue oxygenation. Positive end-expiratory pressure (PEEP) is added to prevent end-expiratory alveolar collapse—this is set at 5 cm H2O for most patients and 20 cm H2O for ARDS patients. Peak flow rates are usually set at 60 L/min. The fraction of inspired oxygen (FiO2) is the percent of oxygen mixed into the inspired gas. The lowest fraction necessary to sustain oxygenation is normally used to prevent oxygen toxicity. FiO2 is titrated to maintain arterial oxygen pressure (PaO2) greater than 60 mmHg and oxygenation saturation (SpO2) above 90%. ARDS patients typically have PaO2 targets 55-80 mmHg and SpO2 targets of 88-95% to reduce plateau pressures and risk of lung injury. While useful in many cases, MV relies on the ability of the lungs and the natural alveolus to exchange oxygen with carbon dioxide in the patient's blood, which may be insufficient in cases where oxygen exchange has been decreased by damage to the lung and/or because of increased stress caused to the injured lung by the treatment. For example, MV may be ineffective for patients experiencing severe hypoxemia arising from lung injury owing to limited mass transfer in the injured lung; over-inflation, barotrauma and/or cyclic closing, and reopening of the alveoli may further damage the lung and trigger a pulmonary and systemic inflammatory reaction that may lead to multiple system organ failure.

Another alternative oxygen supply technique is extracorporeal membrane oxygenation (ECMO), which is a temporary artificial extracorporeal support of the respiratory system and/or cardiac system. First used in adults in 1972 to treat severe respiratory failure (and in 1974 on the first newborn patient), ECMO is a treatment that uses a pump to remove blood from a patient's body, pushes the blood through an external artificial lung to oxygenate the blood and remove carbon dioxide, and then warms the oxygenated blood which is then pumped back into the patient's body. While EMCO is able to augment and/or bypass the need for injured lungs to deliver oxygen and/remove carbon dioxide from the body, this procedure is highly invasive, entails a high risk of thrombosis and contamination of the blood because the blood is removed from the body, and is associated with significant complications and/or adverse side effects. Additionally, ECMO is expensive and complex to operate, limiting its accessibility for emergency care.

Because of the high mortality rate, methods of bypassing the lungs and delivering oxygen directly to the body have been explored for many years. As one example, researchers have attempted to deliver oxygen directly to a patient's bloodstream through intravenous (IV) injection of Oxygen Micro Bubbles (OMB). However, IV injection of OMBs currently appears to be a “one-way” administration that can supply some oxygen to the blood stream and body tissues, but which does not provide for the removal of microbubbles, excess saline and carrier liquids and/or waste carbon dioxide from the blood. Because a patient's bloodstream can only absorb a limited additional volume of blood and/or blood substitutes before hypervolemia or similar conditions occur (i.e., fluid overload), and the direct removal of excess blood fluids from the patient in a safe and effective manner are not yet perfected, the prolonged continuous infusion of oxygen microbubbles and associated carrier liquids into the bloodstream poses significant challenges for clinical translation, including the potential for embolism, thrombosis, hypervolemia, immunogenicity and toxicities of lipid and/or polymer and saline load. Moreover, oxygen which may be inspired through the lungs can potentially be absorbed by the IV microbubbles, which may create long-circulating bubbles that may cause embolism or other problematic conditions, such as those observed in decompression sickness. Further, with the potential for embolism, IV injection of microbubbles requires a strict upper limit on the microbubble size (<10 micrometers) and on the microbubble volume fraction (<70%). Still another problem with IV injection of OMBs is that any nitrogen inspired through the lungs, such as that found in air, will be absorbed by the microbubbles. Thus, the microbubbles will exchange oxygen for nitrogen. The nitrogen-containing microbubbles will be persistent, which can lead to serious problems, such as those observed during decompression sickness and embolism (thus leading to severe morbidity and death).

Another attempt to deliver oxygen to a patient's bloodstream has been through in situ extrapulmonary ventilation (EV), which has successfully oxygenated blood in situ by circulating fluorocarbons, blood, and liposome-encapsulated hemoglobin (a synthetic oxygen carrier) through the intraperitoneal (IP) space, or cavity, of patients. Aside from the highly invasive nature of such surgical treatments, with their attendant risk of complications and/or infection, many of the carriers for delivery of oxygen using these methods are neither safe nor economical. For example, PFCs are expensive to generate and evaporate into potent greenhouse gases creating a significant environmental concern. They are also very stable, tending to accumulate in biological systems in which they are used. Blood and products derived from blood (like synthetic hemoglobin carriers) suffer from scarcity and are relatively expensive to fabricate and store. Furthermore, EV ventilation requires high volumes of perfusate; therefore, a fluid that is economical and biodegradable is important. In addition, this procedure requires the creation of a surgical access into the patient's peritoneum, which can be accompanied by the many attendant risks of abdominal surgery, including infection risk.

Unfortunately, none of the previously developed methods have achieved certain components for extrapulmonary respiration, including (1) delivery of an adequate supply of oxygen for a variety of purposes, including short and/or long term oxygenation, (2) short and/or long-term safety for the patient, (3) ease of administration and/or (4) cost-effectiveness. A demand therefore exists for a system and method for delivery of oxygen to a subject that is more effective and efficient than the current systems and methods presently available.

### BRIEF SUMMARY OF THE INVENTION

The present invention includes the realization of an urgent need for oxygen delivery systems, devices, techniques and/or methods that may partially and/or fully bypass traditional methods of oxygen delivery and/or carbon dioxide removal via the lungs and/or alveoli, especially systems, devices, techniques and/or methods that are non-invasive, easily portable and/or that can quickly deliver oxygen and/or other compounds to a patient or other individual for short term and/or long term delivery and in a safe and easily-used manner.

In various embodiments, the oxygen delivery systems, devices, techniques and/or methods may provide relatively larger quantities of oxygen and/or carbon dioxide removal, including amounts sufficient to fully oxygenate an individual, such as where a patient is experiencing complete cardiopulmonary failure and/or where an individual is located within an anoxic or near-anoxic environment. In other embodiments, the oxygen delivery systems, devices, techniques and/or methods described herein may provide “supplemental” oxygenation and/or carbon dioxide removal for an individual, such as where a patient may be suffering from a lung or other function deficit (but potentially not a total loss of function), or where the individual may be located in a hypoxic environment (i.e., low oxygen conditions). Moreover, the various oxygen delivery systems, devices, techniques and/or methods described herein may be particularly useful in supplementing the blood oxygen demand of various individuals and/or professions, including athletes, pilots, underwater divers, firemen, etc.

In various exemplary embodiments, a method of providing supplemental oxygenation to an individual can include the ingestion and/or other application of microbubbles containing oxygen and/or other substances (including oxygen microbubbles or OMBs) to portions and/or sections of the mucosa of the individual—primarily mucosa of the individual's digestive tract. The oxygen microbubble (OMB) carrier may comprise oxygen filled bubbles having a shell composed of an amphiphilic surfactant phospholipid monolayer or cross-linked polymers or a combination of phospholipids and polymers, and may include other substances to enable and/or facilitate transfer of gases and/or other compounds into and/or out of the microbubbles. Through circulation of oxygen microbubbles through the body cavity, oxygen and carbon dioxide exchange may occur. Overall improvement in extending survival rate time during emergency situations caused by pulmonary or similar oxygen-intake restricting injury and/or failure may be achieved through use of the invented system and methods.

In various embodiments, the OMB formulation may include compounds and/or other features which “target” and/or otherwise demonstrate a preference for various cell types and/or regions of the digestive system for delivery of one or more OMB payloads, including oxygen. For example, dietary nitrate is also an important source of nitric oxide in mammals. Green, leafy vegetables and some root vegetables (such as beetroot) have high concentrations of nitrate. When eaten and absorbed into the bloodstream, nitrate is concentrated in saliva (about 10-fold) and is reduced to nitrite on the surface of the tongue by a biofilm of commensal facultative anaerobic bacteria. This nitrite is swallowed and reacts with acid and reducing substances in the stomach (such as ascorbate) to produce high concentrations of nitric oxide. The purpose of this mechanism to create NO is thought to be both sterilization of swallowed food, to prevent food poisoning, and to maintain gastric mucosal blood flow. In at least one exemplary embodiment, the OMB formulation may include an additional compound, such as nitric oxide and/or compounds which break down in the human body to form nitric oxide and/or its analogues, to desirably promote gastric mucosal blood flow and/or transferal of oxygen/carbon dioxide between the OMB's and the surrounding anatomy/blood flow.

In various embodiments, the OMBs may deliver oxygen to one or more specific locations within the digestive tract, or the delivery of oxygen and/or other compounds may occur at multiple locations and/or along the entirety of the digestive tract and/or various portions thereof. Where an individual OMB has delivered some portion of its oxygen payload (and/or other compounds) to a portion of the individual's anatomy, the individual OMB may “destruct” (i.e., “popping” of the microbubble), the OMB may reduce in size to become a smaller microbubble, and/or the OMB may increase in size via absorption and/or incorporation of other substances (i.e., carbon dioxide, other gases and/or metabolic wastes).

In various embodiments, the amphiphilic phospholipid monolayer shell variation of an exemplary OMB embodiment can have similar composition to lung surfactant and may require comparable physical properties, such as rapid adsorption to and mechanical stabilization of the gas/liquid interface and high gas permeability. Thus, OMBs can be designed to mimic the mechanical and gas transport properties of the alveolus to deliver the oxygen payload. By transport through the digestive tract, phospholipid monolayer, cross-linked polymer or mixed phospholipid-polymeric stabilized OMBs will desirably provide oxygen for uptake through tissues to the bloodstream. In addition, any “unused” microbubbles can easily pass through and/or be removed from the digestive system, including by elimination in the natural progression, as well as any component materials from OMBs that “burst” or otherwise destruct or are released during such activities.

### BRIEF DESCRIPTION OF THE SEVERAL VIEWS OF THE DRAWINGS

FIG. 1 graphically depicts one exemplary embodiment of oxygen microbubbles;

FIG. 2 graphically depicts exemplary microbubble oxygen content over time;

FIG. 3 graphically depicts exemplary results from an OMB formulation consumption test by an adult male cyclist operating a stationary bicycle over a period of time;

FIG. 4A graphically depicts partial enlarged views of comparison graphs of conditions prior to OMB ingestion and after OMB ingestion;

FIG. 4B graphically depicts a comparison and analysis of the pre and post-OMB test of FIG. 4A;

FIG. 5 graphically depicts exemplary results from a water OMB formulation consumption test by an adult male cyclist operating a stationary bicycle over a period of time;

FIG. 6 graphically depicts exemplary results from a water supersaturated with oxygen formulation consumption test by an adult male cyclist operating a stationary bicycle over a period of time;

FIG. 7 graphically depicts exemplary results of an alternative OMB formulation ingestion test; and

FIG. 8A graphically depicts the heart rate information of the cyclist from the tests of FIGS. 5, 6 and 7;

FIGS. 8B and 8C graphically depict an average heart rate and a normalized heart rate change, respectively, of the cyclist from 12.5 minutes to 25 minutes across the three tests of FIGS. 5, 6 and 7;

FIGS. 9A and 9B graphically depict absolute and normalized heart rate comparisons for pre and post-OMB conditions; and

FIG. 10 graphically depicts heart rate data from sensors showing a lower heart rate increases over a 10-minute window post-OMB ingestion.

### DETAILED DESCRIPTION OF THE INVENTION

The drawings and the following description relate to preferred embodiments by way of illustration only. It should be noted that from the following description, alternative embodiments of the components and methods disclosed herein will be readily recognizable as viable alternatives that may be employed in one skilled in the art.

In various exemplary embodiments, a method of providing supplemental oxygenation to an individual can include the ingestion and/or application of microbubbles containing oxygen and/or other substances (including oxygen microbubbles or OMBs) to portions and/or sections of the mucosa of the individual. The oxygen microbubble (OMB) carrier may comprise oxygen filled bubbles having a shell composed of an amphiphilic surfactant phospholipid monolayer, a cross-linked polymer, or a combination of phospholipids and polymers. In one variation, the amphiphilic phospholipid monolayer shelled OMB can have a similar composition to lung surfactant and requires comparable physical properties, such as rapid adsorption to and mechanical stabilization of the gas/liquid interface and high gas permeability. Thus, OMBs can be also designed to mimic the mechanical and gas transport properties of the alveolus to deliver the oxygen payload. By transport through the digestive tract, phospholipid monolayer OMBs will desirably provide oxygen for uptake through tissues to the bloodstream. Similarly, biocompatible polymer shelled microbubbles can readily be delivered to the digestive track via ingestion or injection and are able to deliver oxygen throughout the entire track and be naturally removed from the anatomy.

The mucosa is a mucous membrane which lines the inside of the digestive tract of an individual from mouth to anus, and depending upon the “section” of the digestive tract, the mucosa protects the digestive tract wall, secretes substances, and has the capability to absorb various substances, including the end products of digestion. In an adult human, the total surface area of the mucosa has been estimated from 30 to 40 square meters (in some studies) and/or up to approximately 400 square meters (in other studies), which is near to and/or far exceeds the 50 to 75 square meter surface area of the lungs. In various embodiments, OMB's may be ingested or otherwise introduced into an individual's body (i.e., via a stomach or intestinal feeding tube and/or enema, for example) where the OMBs may contact the mucosa and/or other tissues and may transfer a gas, compound and/or other payload into and/or through the cells of the mucosa for local treatment and/or systemic treatment and/or distribution via the blood stream and/or lymphatic system.

Phospholipid monolayer microbubbles may be used in combination with other gases and additives to provide an optimum composition for specific physiologic effects. Anesthetic gases delivered by oral ingestion of phospholipid monolayer microbubbles may: provide enhanced anesthetic saturation levels for mammals with compromised lung function; provide enhanced anesthetic performance by delivery of anesthetic agents to the body. In various embodiments, a variety of anesthetic compounds may be delivered in conjunction with the OMB formulation, which may include substances to augment anesthetic compounds provided for certain medical purposes as well as agents that may enable and/or enhance anesthetic effects for pain relief, surgical interventions, dental treatments, and relief of physical discomfort.

According to the invention, OMBs can be designed for high oxygen carrying capacity, high oxygen delivery rate and sufficient stability for storage and transport. Direct systemic oxygenation by introducing OMBs into the digestive tract and/or other naturally-existing cavities of the body (i.e., using pre-existing natural access openings) is a radical change from existing oxygen delivery platforms. In various embodiments, the OMBs may circulate naturally within the digestive tract and may even be digested and/or naturally eliminated by the body. Desirably, this therapy will preclude the need for an extracorporeal loop to circulate blood, thus potentially circumventing the complications from thrombosis and intracranial hemorrhage presented by ECMO.

As used herein, microbubbles generally refer to micron-sized (e.g., in the range of 1 um to 1000 um in diameter) substantially-spherical gas-filled particles in solution that are stabilized by an organic coating at the gas-liquid interface. The stability, gas diffusion properties, and biocompatibility of microbubbles can be controlled via the formulation of the coating material (i.e., the microbubble shell). Customizing the stabilizing shell of the microbubbles can allow fabricated microbubbles to be stored for later use. Alternatively, the microbubbles may be used immediately after fabrication. In such cases, the coating material may be sufficiently stable as to allow the microbubble to deliver its gas payload to an intended target (e.g., digestive tract of a patient).

According to various features of the present invention, OMBs can be designed and constructed for high oxygen carrying capacity, high oxygen delivery rate and/or sufficient stability for storage and transport. Direct systemic oxygenation by oral ingestion of OMBs into the digestive tract is a radical change from existing oxygen delivery platforms. The procedure for delivery of OMBs through the digestive tract is simple and straightforward, and the therapy precludes the need for an extracorporeal loop to circulate blood, thus potentially circumventing the complications from thrombosis and intracranial hemorrhage presented by ECMO.

Since blood is oxygenated by OMBs administered through the digestive tract, the infusion of OMBs directly into circulation is avoided. In addition, the upper size limit (about 10 um diameter) is not required to avoid vascular occlusion. In certain embodiments, larger microbubbles (about 10-25 um diameter) can be perfused through the digestive tract without fear of adverse effects because they are separated by gas permeable membranous tissue from the intravascular space. Thus, the effects of larger microbubble size distributions on microbubble suspension viscosity and oxygen-release rate (at equivalent volume fraction) may be measured. According to the invention, microbubbles may be between 1-25 um in diameter with larger microbubbles being about 9-25 um in diameter and smaller microbubbles being about 1-8 um in diameter. However, it is contemplated that microbubbles may be between 1-100 um in diameter and even between 1-500 um in diameter. In addition, mixtures of microbubbles may comprise microbubbles of different sizes. The sizes of the OMBs contained within any one mixture may be only smaller microbubbles, only larger microbubbles or a combination of both smaller and larger microbubbles.

In various embodiments, the oral or other delivery of a gas contained within a phospholipid and/or polymeric monolayer shell microbubble may include gases other than oxygen, or in combination with oxygen, including nitrogen, hydrogen, fluorine or fluorinated gases, chlorine, helium, neon, argon, krypton, xenon and/or radon in varying compositions according to the desired therapeutic effect. Hyperoxic mixes may be used as a means to draw dissolved inert gases from the body. In other embodiments, the microbubbles may include gaseous compounds other than oxygen, or in combination with oxygen or other elements, including NO2 (nitrous oxide), CO2 (carbon dioxide) CH4 (methane), NH3 (ammonia), HCN (hydrogen cyanide), CO (carbon monoxide), NO (nitrogen oxide), C2H6 (ethane), PH3 (phosphine), H2S (hydrogen sulfide), HCl (hydrogen chloride), CO2 (carbon dioxide), N2O (dinitrogen oxide), C3H8 (propane), NO2 (nitrogen dioxide), O3 (ozone), C4H10 (butane), SO2 (sulfur dioxide), BF3 (boron trifluoride, Cl2 (chlorine), CF2Cl2 (dichlorodifluoromethane) and/or SF6 (sulfur hexafluoride) in varying compositions according to the desired therapeutic effect.

The ability to deliver oxygen via OMB ingestion may also have significant clinical implications. For example, acute severe hypoxia of any origin (due to airway obstruction or due to other causes) generally results in irreversible brain injury within minutes. Administration of readily accessible oxygen-bearing microbubbles may prevent the morbidity and mortality associated with acute hypoxia in many subjects such as human subjects. In addition, subjects suffering from lung injury, which represent a significant percentage of those requiring intensive care, may benefit from the delivery of oxygen that offers minimally invasive extrapulmonary oxygen supplementation. Thus, ventilator-induced lung injury may be minimized while avoiding critical hypoxia. Increasing systemic oxygen saturations may improve hypoxic pulmonary vasoconstriction and reduce pulmonary vascular resistance in subjects with acute exacerbations of pulmonary hypertension. Infants, for example, born with cyanotic congenital heart disease, could benefit from an effective delivery of oxygen that may lessen their hypoxemia preoperatively, as well as during the early postoperative recovery period. This therapy could also provide care for cases of irreversible pulmonary failure and, hence, act as a bridge to lung transplant.

In various embodiments, the delivery of supplemental oxygen in lower concentrations via OMB ingestion (i.e., less than 25% of physiologic demand or less than 20% of physiologic demand or less than 15% of physiologic demand or less than 10% of physiologic demand or less than 5% of physiologic demand or less than 4% of physiologic demand or less than 3% of physiologic demand or less than 2% of physiologic demand or less than 1% of physiologic demand) may be particularly useful immediately prior to and/or during the onset of transient and/or progressively worsening medical conditions that may affect the individual's breathing and/or blood oxygenation levels. For example, the ingestion and/or other intake (i.e., inhalation) of an OMB formulation may be particularly helpful prior to and/or during the onset of a severe asthma attack, which can include physiological effects (i.e., by increasing blood oxygenation levels and/or supplying oxygen directly to critical tissues in the body) as well as psychological effects (i.e., by making the patient feel less suffocated” or “out of breath”). Various combinations of such effects may provide significant clinical improvement for such individuals by reducing stress and/or otherwise alleviating the attack, or if drunk in earlier stages, the OMB oxygen could provide significant benefits in case the attack progresses to a severe stage.

Similarly, the delivery of supplemental oxygen in lower concentrations via OMB ingestion may be particularly useful for individuals anticipating a significant increase in their oxygen demand and/or who anticipate that they will be encountering hypoxic and/or anoxic conditions, such as performance athletes preparing for an athletic activity and/or individuals attempting to escape from a building fire or airline passengers or pilots suffering from atmospheric depressurization. In such cases, the OMB formulation may be consumed prior to such event, and serve as an oxygen “reservoir” that can be tapped by the body at need for a limited duration of time, thereby improving the individual's performance and/ability to function for an increased length of time.

Phospholipid monolayer or cross-linked polymer or phospholipid-polymeric microbubbles may be used in combination with other fluids and additives to provide an optimum composition for specific physiologic effects. Oxygen delivered by oral ingestion of stabilized oxygen microbubbles may: reduce harmful effects of tumors by decreasing hypoxia; provide enhanced oxygen saturation levels for mammals with compromised lung function; provide enhanced athletic performance by delivery of oxygen, sucrose, glucose, caffeine, or other agents to the body; promote healing of wounds, burns, or other injuries where oxygen is of importance to reduced healing or recovery time. In various embodiments, a variety of “performance enhancing” or “replacement” compounds may be delivered in conjunction with the OMB formulation, which may include substances to replace compounds lost, eliminated and/or utilized by the individual during certain activities (i.e., water, sodium, potassium, phosphates, citric acid) as well as substances that may enable and/or enhance absorption of OMB constituents by the mucosa.

In some exemplary embodiments, administration of the OMB formulation may be associated with one or more additional compounds that modify the individual's digestive tract (or portions thereof) to facilitate the durability, passage and/or absorption of, enable and/or facilitate absorption of OMB constituents by the mucosa. For example, it may be desirous to alter the pH level of the stomach prior to and/or during ingestion of the OMB formulation, as the normal acidity levels of the stomach may reduce and/or limit the durability of the microbubbles and/or negatively affect the ability of the OMBs to transfer oxygen through the mucosal walls of the stomach. Such alteration might be accomplished by the ingestion of an antacid formulation (i.e., calcium carbonate tablets) just prior to ingestion of the OMB formulation, or the antacid may be incorporated into the OMB formulation for concurrent and/or subsequent ingestion. In a similar manner, it may be desirous to alter the speed at which the OMB formulation or portions thereof pass through the various stages of the digestive tract, which might include the administration of magnesium citrate or some other intestinal stimulant prior to, concurrent with and/or after ingestion of the OMB formulation.

Oral administration of pharmaceuticals and other therapeutic materials has considerable advantages in terms of patient acceptability, reducing the risk of infection, cost and the quantity of material that can be delivered. Frequently, however, oral administration may be associated with inefficient delivery and/or poor bioavailability, but unlike the administration of oxygen saturated water for reducing recovery times for athletes (the effectiveness of which has little support in the clinical literature), use of shell-stabilized oxygen microbubbles for oral ingestion provides a stable delivery medium which delivers oxygen without compromise of the circulatory system, and with reduced risk of embolism or of thrombogenic complications.

In various embodiments, the disclosed formulations of phospholipid and/or polymer stabilized microbubbles have multiple applications in hypoxia, including: increasing sports endurance; relief of high altitude pulmonary edema; supply of oxygen to ambulatory patients relying on oxygen concentrators; relieving oxygen insufficiency underwater; provide first response oxygen delivery in lung injury, treat smoke inhalation, and trauma, including to provide oxygen supersaturation in advance of medical interventions such as intubation and/or oxygenation in environments which are hostile to inhalation.

In various embodiments, the oral delivery of the OMB formulation desirably overcomes the risks associated with direct intravenous surgical injection of high concentrations of microbubbles (e.g. embolism, clot, etc.) into the patient's body, and oral ingestion delivery should offer improved diffusion characteristics leading to more efficient delivery compared with direct vascular injection, as well as greater patient acceptability. Unlike surfactant and lecithin-based mixtures (which may provide varying levels of effectiveness in various alternative embodiments), using known and isolated amphiphilic phospholipids and biocompatible polymers as the shell material in OMBs desirably provides a mixture composition that is fully understood, thereby allowing for the behavior of the OMBs to be relatively predictable. This enhanced OMB behavior predictability allows the OMBs to be fabricated for greater stability, control of oxygen release, manufacturability, improved storage and handling, and greater efficacy in oxygen delivery. Additionally, OMBs on the order of 1-1000 um in diameter experience a lower internal Laplace pressure (responsible for driving dissolution) than OMBs 1-999 nm in diameter range, allowing the micron-sized OMBs to persist longer in the digestive tract.

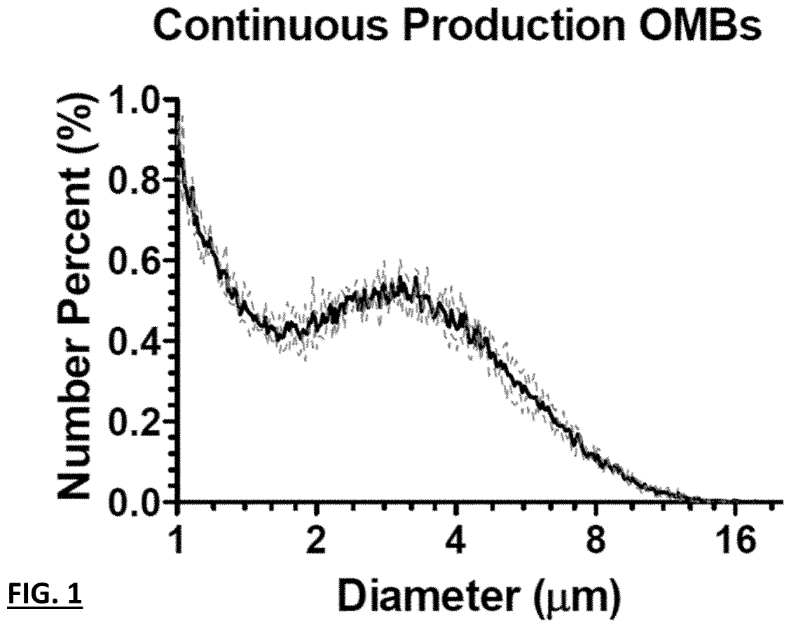

FIG. 1 depicts a graph of one exemplary embodiment of oxygen microbubbles, which can be produced using a variety of production methods and/or techniques, including continuous production and/or batch production. If desired, the OMBs can be produced immediately prior to use, or they can be manufactured and stored for extended periods of time prior to use in the various embodiments described herein.

In at least one exemplary embodiment, the size of the OMBs utilized herein can be primarily distributed between 1 and 10 microns (um) in diameter, although larger and/or smaller microbubbles and/or microbubble distributions can be utilized in a variety of the disclosed embodiments with varying results.

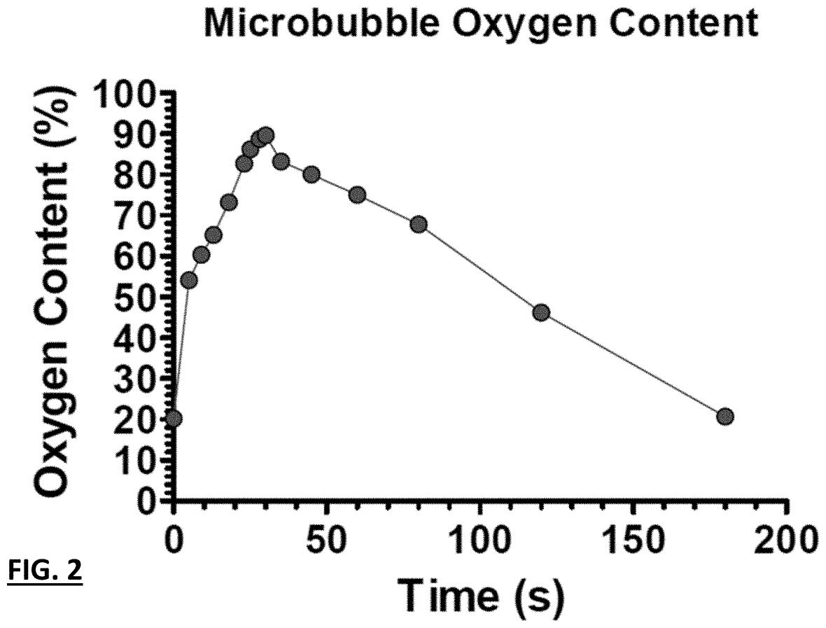

FIG. 2 depicts a graph of microbubble oxygen content over time, specifically an amount of oxygen being released from within phospholipid microbubbles through a diffuse oxygen sensor. For measurement, 10 mL of phospholipid OMBs were broken down in a gas tight syringe via cyclic pressurization. Once full OMB destruction was observed (i.e., no foam, only liquid left in syringe, ^{˜}1-2 mL of liquid volume), the syringe was connected to the diffuse oxygen sensor, allowing the oxygen to pass through the sensor and be measured. The sensor was stored in a natural air environment prior to measurement (^{˜}20% oxygen).

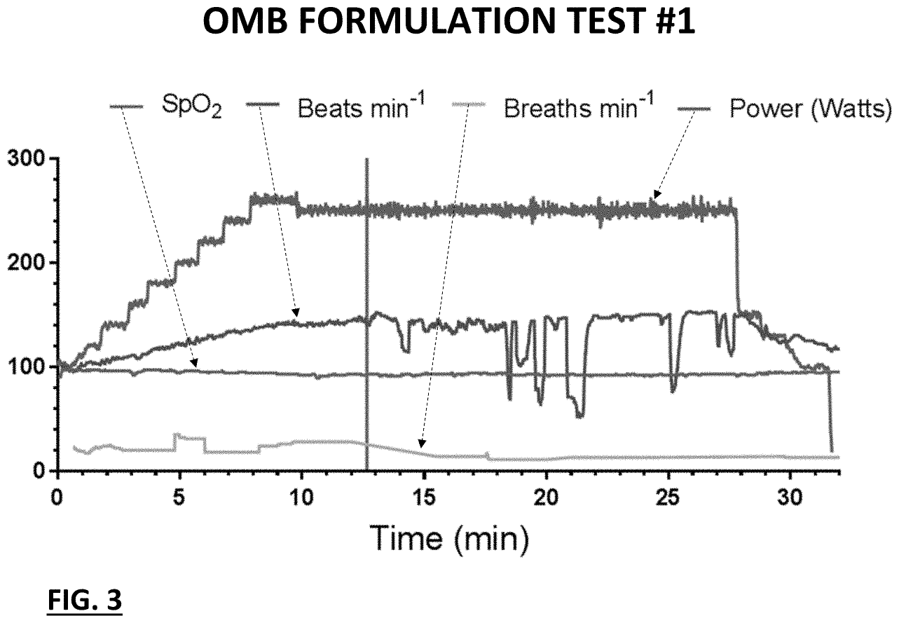

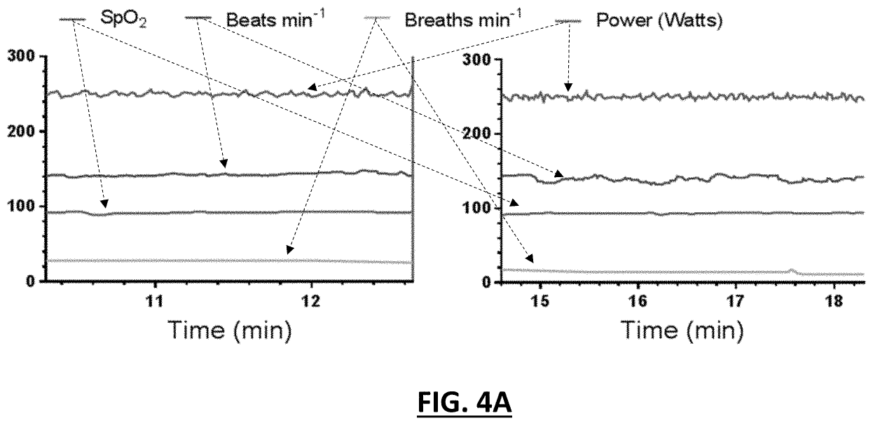

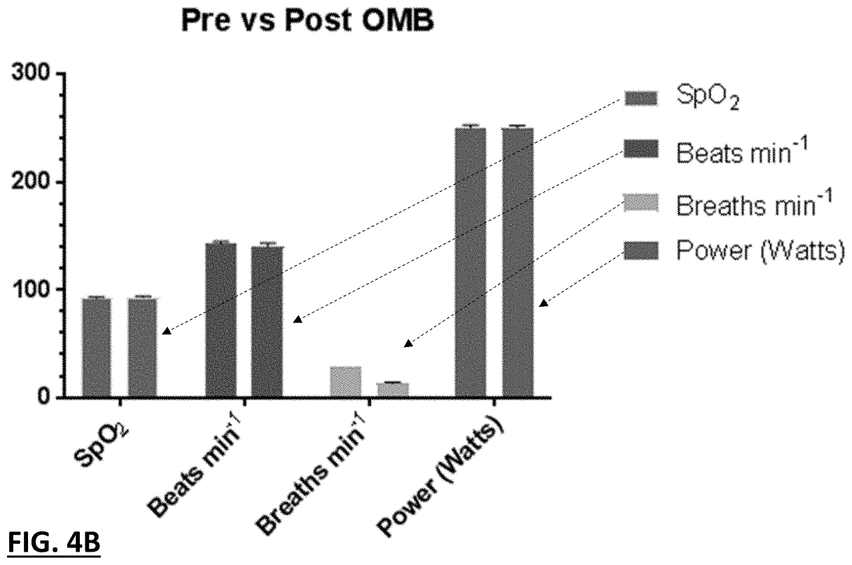

In one exemplary embodiment, a liquified slurry of OMBs was created generating a OMB solution with an approximately 60% void fraction (60% oxygen microbubbles, 40% liquid) within a carrier solution such as saline. The resulting OMB mixture was consumed (i.e., ingested by drinking) from a bottle by an adult male cyclist (90 kg, 30 years old) operating a stationary bicycle, with various vital statistics being monitored and recorded. The cyclist began cycling at 0 seconds, and reached a relatively stable “plateau” level in his exercise routine at 7 minutes of continuous cycling. The OMB formulation was consumed at 12.5 minutes, and the cyclist continued exercising at the plateau levels for an additional 15.5 minutes, then performed a warm-down period of 4 minutes, for a total test time of 32 minutes. The results of this test are depicted in FIGS. 3 and 4A.

FIG. 4A depicts partial enlarged views of two portions comparison graphs of conditions prior to OMB ingestion (on the left), and after OMB ingestion (on the right). It was observed that the cyclist maintained the same output levels prior to and after ingestion of the OMB formulation, with the same heartbeat and oxygen saturation, but the breathing rate of the cyclist significantly dropped within 2 minutes after consumption of the OMBs, and this lower breathing level was maintained for a significant period of time (i.e., at least 10 minutes). Is it demonstrated that the OMB formulation enabled the cyclist to reduce his breathing rate by at least half while maintaining equivalent output levels and maintaining equivalent oxygen levels and heartbeat.

A comparison and analysis of this pre and post-OMB test is provided in FIG. 4B. In this graphic, it can be seen that the output levels, heartbeat and oxygen saturation levels were equivalent pre and post OMB, but the breathing level of the cyclist was significantly lower after ingestion of the OMB formulation. The cyclist reported feeling a brief euphoria at the time of OMB ingestion, which may be at least partially due to the placebo effect (as the cyclist was aware of which drink contained the OMBs) and/or potentially due to the inhalation of pure oxygen released from the headspace of the OMB bottle, or due to perceived reduction in level of effort. The subsequent continuous dramatic reduction in the breathing rate of the cyclist, however, would typically result in decreased oxygen absorption via the lungs into the cyclist's bloodstream (and a commensurate drop in blood O₂ levels), which did not occur. Rather, the cyclist's saturated oxygen levels and heartbeat remained constant, which can be explained by the absorption of oxygen from the ingested OMB into the cyclist's bloodstream via the digestive tract, which is believed to have supplemented the reduced oxygen intake via the cyclists lungs during the extended period of reduced breathing, which continued for many minutes after OMB ingestion.

Another significant finding from this OMB test was that the reduced breathing rate of the cyclist did not appear to cause a significant increase in heart rate, an increase expected with reduced breathing rate. In general, a reduced breathing rate should reduce the rate that carbon dioxide is being removed from the blood, resulting in an increased level of carbon dioxide in the blood (and also an increased acidity of the blood). The amount of carbon dioxide in the blood typically exerts a strong influence on the respiratory rate, and as the activity level increases, cells—particularly muscles cells—produce increased amounts of carbon dioxide. The rhythmicity center in the brainstem detects increased carbon dioxide and increases the respiratory rate to eliminate the excess via the lungs. In addition, sensors called the aortic and carotid bodies detect changes in blood pH, with the lungs and kidneys collaboratively controlling blood pH, such that an abnormally low blood pH typically increases the respiratory rate in an involuntary manner. In the present test, however, the exact opposite occurred, leading to the hypothesis that the OMB formulation is also removing and/or facilitating removal of some additional amount of carbon dioxide from the blood of the cyclist after ingestion, thereby allowing the cyclist to breath at a lower rate without experiencing a rapid and unwanted accumulation of carbon dioxide in his blood stream.

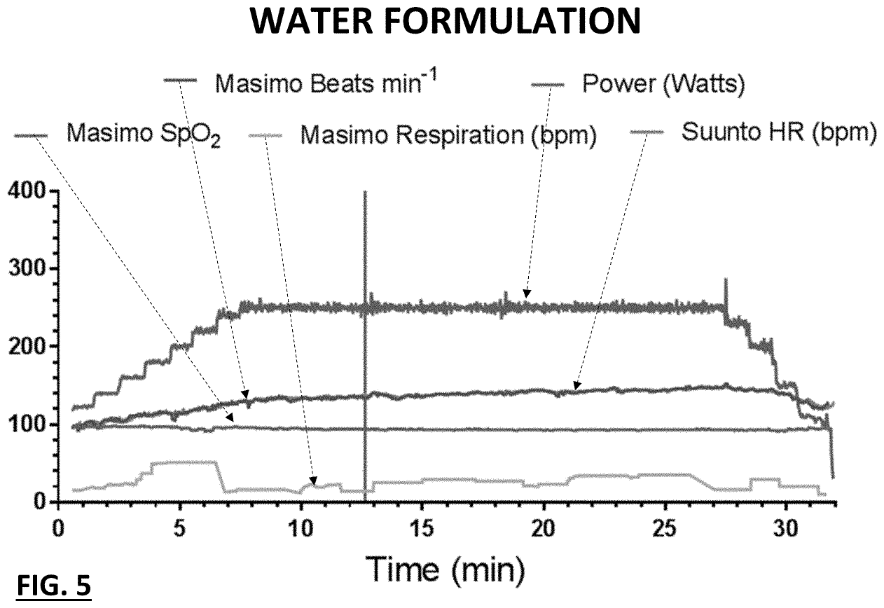

FIG. 5 depicts a control experiment conducted on the same adult male cyclist with ingestion of a water formulation. This test showed that the cyclist could maintain a consistent output level prior to and after ingestion of the water formulation, but the breathing rate of the cyclist was generally consistent during the full length of this test and/or increasing after ingestion of the water formulation, along with an expected increase in the heartbeat level and a slight reduction in blood O2 saturation over the 32 minute test period.

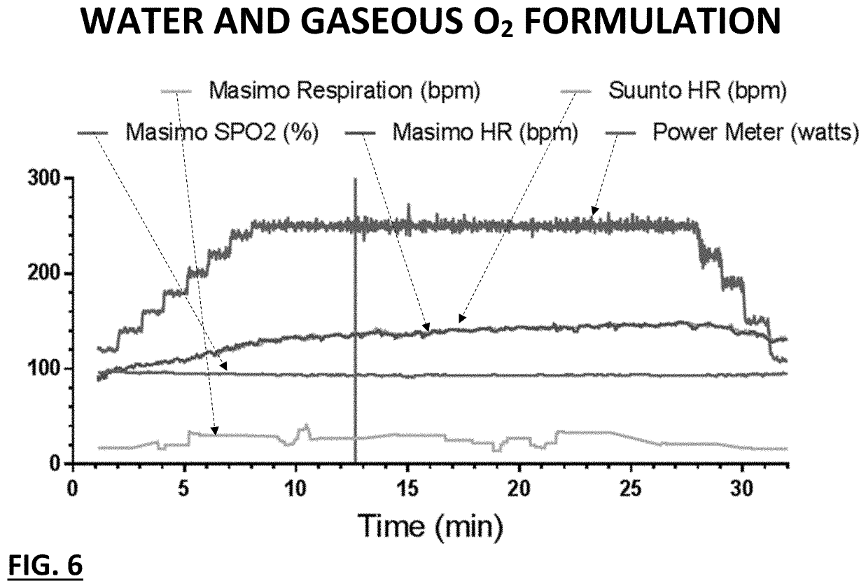

FIG. 6 depicts another control experiment conducted on the cyclist using a water formulation supersaturated with gaseous oxygen (″O₂ water). This test showed that the cyclist could maintain a consistent output level prior to and after ingestion of the O₂ water formulation, with the breathing rate of the cyclist generally consistent during the test, along with a constant increase in the heartbeat level and a consistent blood O₂ saturation level over the 32 minute test period. The results of this test show little effect, if any, from the gaseous oxygen within the water, which infers that the O₂ was not absorbed by the cyclist in significant amounts during this test.

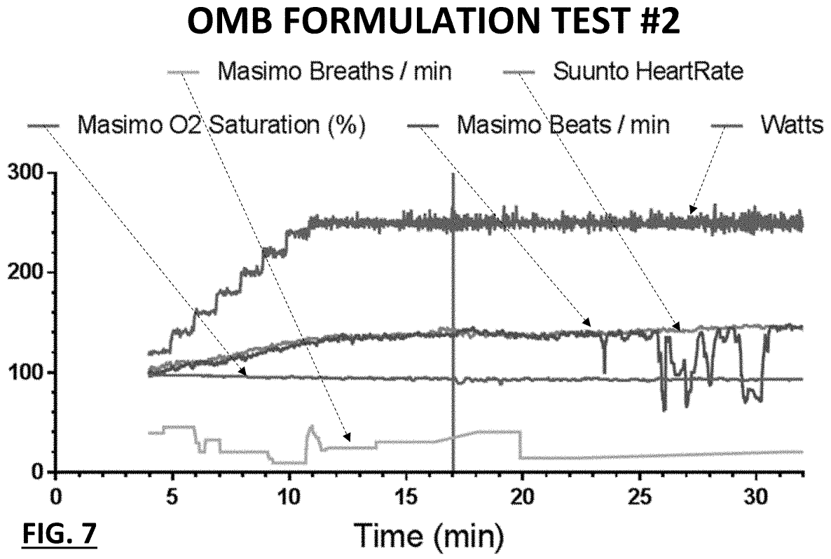

FIG. 7 graphically depicts the results of a second OMB formulation ingestion test, using the same adult male cyclist and test conditions as Test #1. During this test, a similar transient reduction in the breathing rate of the cyclist was recorded approximately 2.5 minutes after ingestion of the OMB formulation, with no significant change in the cyclist's heartrate and/or O2 blood saturation for the remainder of the 32 minute test, which confirmed the hypothesis that absorption of oxygen from the ingested OMB into the cyclist's bloodstream via the digestive tract was occurring for an extended period of time after OMB ingestion.

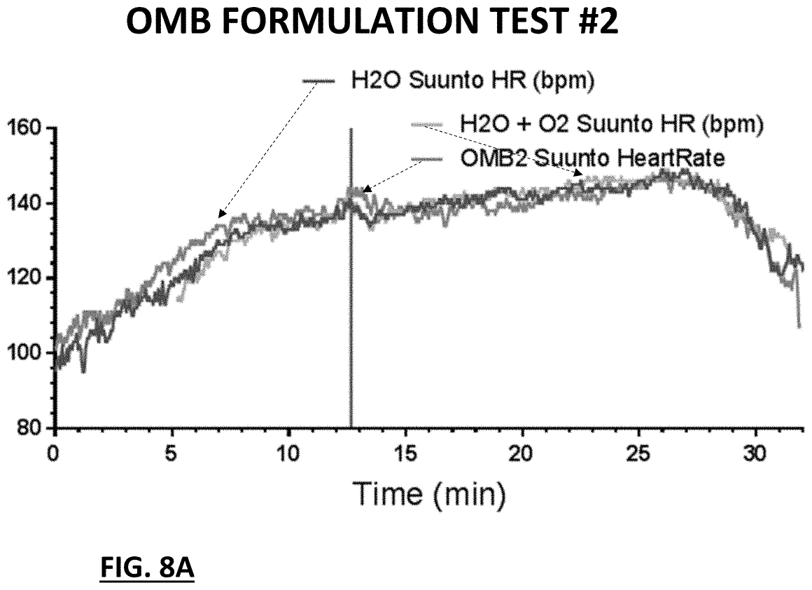

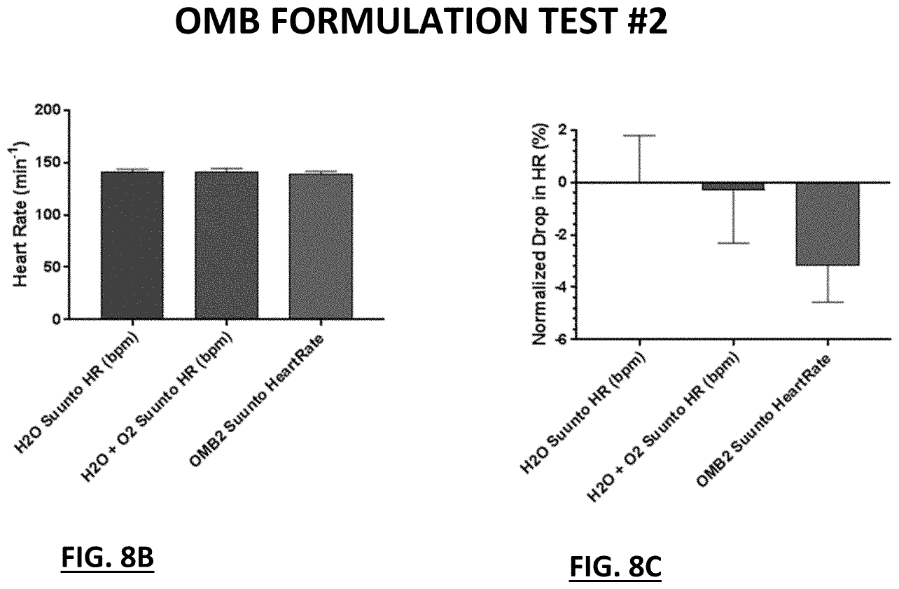

FIGS. 8A through 8C, 9A, 9B, and 10 graphically depict additional information obtained during the second OMB ingestion test. Specifically FIG. 8A shows the heart rate of the cyclist for when he ingested water, water with an oxygen headspace, and oxygen microbubbles. It can be seen that for all three datasets that the subjects heart rate increased temporarily at the time of administration. Interestingly, the subjects heart rate during the OMB trial declined slightly and was maintained at a rate lower than the water or water+oxygen gas trials.

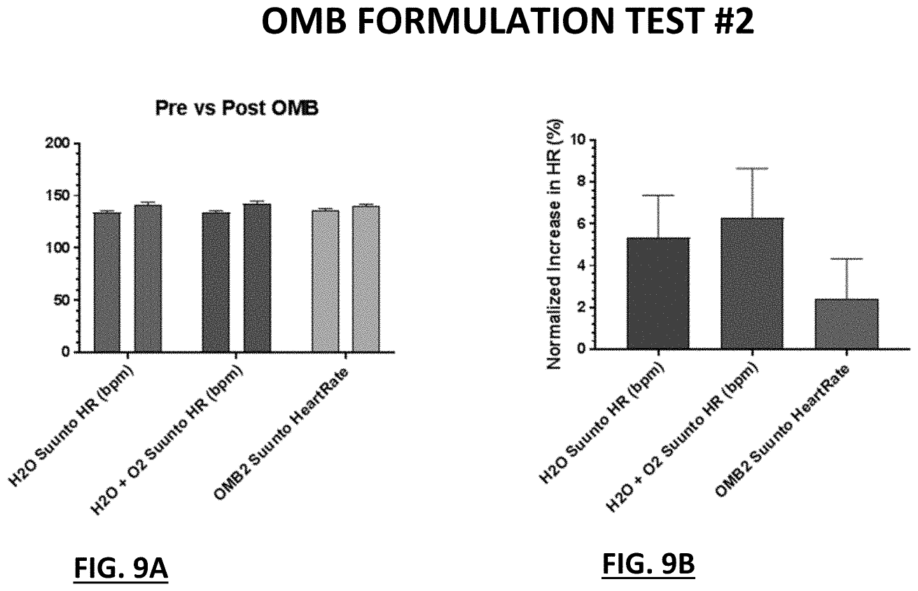

As best seen in FIG. 8B, the test results show that the average heart rate from 12.5 minutes to 25 minutes is nearly the same across all three trials, but FIG. 8C shows that the OMB formulation provided the greatest drop in heart rate from the time of administration compared to the 15 to 25 minute averaged window. Similarly, FIGS. 9A and 9B show that ingestion of the OMB formulation led to the lowest increase in heart rate as compared to the water and water/O₂ mixtures.

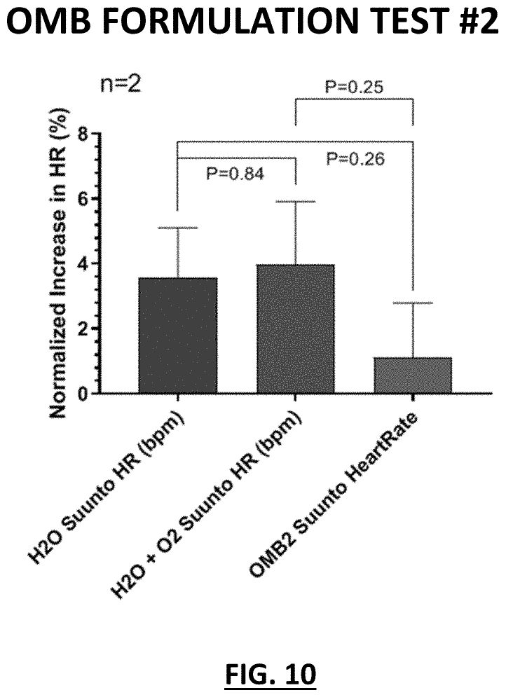

As best seen in FIG. 10, the heart rate data from the Masimo and Suunto monitors (n=2) shows that ingestion of the OMB formulation may reduce the amount of heart rate increase over a 10-minute window post-ingestion. Additional test results may least to statistically significant results (i.e., n=3 or greater)

Increased Awareness/Placebo Effect/Dual Mode Oxygen Delivery

In at least one exemplary embodiment, the oral consumption of an OMB formulation can potentially provide “concurrent loading” of oxygen delivery for an individual at the time of consumption because: (1) some amount of the oxygen in the OMBs may be quickly absorbed in the mouth (i.e., sublingual space), throat, trachea and/or stomach of the consuming individual, immediately increasing the level of oxygen in the blood stream of the individual (including the potential for localized blood oxygen increases in specific anatomical areas such as the head and/or heart of the individual), (2) some of the OMB material may remain resident in the airway after ingestion, which can temporarily increase the concentration of oxygen being drawn into the airways and lungs of the patient, and (3) the OMB formulation may contain a significant amount of “free” oxygen in the “headspace” of the container, which oxygen may be breathed in by the individual. This may result in a temporary feeling of mental clarity or increased awareness for a limited period of time. For example, during a test at an altitude of approximately 10,500 feet (SpO2=^{˜}80%), the test subject felt a significant mental response immediately after drinking approximately 300 mL of an OMB formulation. While it was possible that a placebo effect contributed to this feeling, it was also suggested that the pure oxygen in the container headspace had been inhaled at the same time as ingesting the OMB.

Blood pH—Acidosis and Alkalosis

An important property of blood is its degree of acidity or alkalinity. The acidity or alkalinity of any solution, including blood, is indicated on the pH scale. The pH scale, ranges from 0 (strongly acidic) to 14 (strongly basic or alkaline). A pH of 7.0, in the middle of this scale, is neutral. Blood is normally slightly basic, with a normal pH range of 7.35 to 7.45. Usually the body maintains the pH of blood close to 7.40, and the body's balance between acidity and alkalinity is referred to as acid-base balance. The blood's acid-base balance is precisely controlled because even a minor deviation from the normal range can severely affect many organs. The body uses different mechanisms to control the blood's acid-base balance. These mechanisms involve the lungs, the kidneys and various buffer systems.

One mechanism the body uses to control blood pH involves the release of carbon dioxide from the lungs. Carbon dioxide, which is mildly acidic, is a waste product of the processing (metabolism) of oxygen and nutrients (which all cells need) and, as such, is constantly produced by cells. It then passes from the cells into the blood. The blood carries carbon dioxide to the lungs, where it is exhaled. As carbon dioxide accumulates in the blood, the pH of the blood decreases (acidity increases).

The brain regulates the amount of carbon dioxide that is exhaled by controlling the speed and depth of breathing (ventilation). The amount of carbon dioxide exhaled, and consequently the pH of the blood, increases as breathing becomes faster and deeper. By adjusting the speed and depth of breathing, the brain and lungs are able to regulate the blood pH minute by minute. The kidneys are able to affect blood pH by excreting excess acids or bases. The kidneys have some ability to alter the amount of acid or base that is excreted, but because the kidneys make these adjustments more slowly than the lungs do, this compensation generally takes several days. Yet another mechanism for controlling blood pH involves the use of chemical buffer systems, which guard against sudden shifts in acidity and alkalinity. The pH buffer systems are combinations of the body's own naturally occurring weak acids and weak bases. These weak acids and bases exist in pairs that are in balance under normal pH conditions. The pH buffer systems work chemically to minimize changes in the pH of a solution by adjusting the proportion of acid and base. The most important pH buffer system in the blood involves carbonic acid (a weak acid formed from the carbon dioxide dissolved in blood) and bicarbonate ions (the corresponding weak base).

Acidosis and alkalosis are categorized depending on their primary cause as metabolic and respiratory. Metabolic acidosis and metabolic alkalosis are caused by an imbalance in the production of acids or bases and their excretion by the kidneys, while respiratory acidosis and respiratory alkalosis are caused by changes in carbon dioxide exhalation due to lung or breathing disorders. Regardless of source, however, each acid-base disturbance a patient experiences typically provokes automatic compensatory mechanisms that push the blood pH back towards normal. In general, the respiratory system compensates for metabolic disturbances while metabolic mechanisms compensate for respiratory disturbances. At first, the compensatory mechanisms may restore the pH close to normal. Thus, if the blood pH has changed significantly, it means that the body's ability to compensate is failing. In such cases, doctors urgently search for and treat the underlying cause of the acid-base disturbance.

In various embodiments, ingestion and/or introduction of an OMB formulation into a body cavity of a patient may assist with the management of acidosis and/or alkalosis, in that the OMBs are capable of absorbing carbon dioxide from an individual, in a manner similar to the release of carbon dioxide from the lungs, and the microbubbles containing the removed carbon dioxide can be excreted and/or removed from the patient in various manners.

According to the invention, OMBs can be introduced into the digestive tract of an individual and allowed some time to deliver oxygen and absorb carbon dioxide, followed by the intentional removal and/or natural elimination of the OMBs to remove them from the digestive tract, followed by another introduction of OMBs into the body. If desired, this process cycle can be is repeated as necessary. In the alternative, OMBs may be continuously circulated through a cavity within the digestive tract to release oxygen and absorb carbon dioxide and other gases.

Drug Delivery

In various embodiments, the ingestion of OMBs and/or other microbubble formulations may enhance and/or facilitate the delivery and/or absorption of oxygen (or reverse transfer of carbon dioxide) and/or may enhance and/or facilitate the delivery of other compounds and/or medications in local and/or systemic manners. For example, OMBs and/or other microbubble formulations may be particularly useful in delivering cannabinoids and/or similar substances to an individual, including the psychoactive Δ⁹-tetrahydrocannabinol (THC) and the non-psychoactive cannabidiol (CBD), commercially available as pharmaceutical formulations such as Nabiximols (Sativex®—a commercially available oromucosal spray that contains a mixture of THC and CBD) and Dronabinol) (Marinol®), an oral preparation of synthetic THC. In addition, the phospholipid monolayer variation of microbubbles described herein may have particular affinity and usefulness in conjunction with the lipid-soluble cannabinoids THC and CBD, as the oral co-administration of lipids may increase bioavailability of THC in mammals by more than 2.5-fold, and of CBD by almost 3-fold (which profound increase in systemic exposure may significantly affect the therapeutic effects or toxicity of these cannabinoids).

In various embodiments, the microbubbles may be constructed similarly and/or act similarly to chylomicrons within a body. A small fat globule composed of protein and lipid (fat), chylomicrons are found in the blood and lymphatic fluid where they serve to transport fat from its port of entry in the intestine to the liver and to adipose tissue. Since the lipid composition of lipid-shell microbubbles can be similar to that of chylomicron remnant particles, the active uptake of the chylomicron remnant particles by their associated “lipoprotein receptor”—mediated endocytic pathways may induce a similar active-uptake pattern for microbubbles. Accordingly, in various embodiments much of the microbubble size distribution could be in the same diameter range as observed with chylomicron remnants. Although natural chylomicrons (CM) typically have much more complex structure than OMBs and similar microbubbles, the uptake of lipophilic compounds by artificial emulsions has been shown to provide a reasonably close estimate for the degree of association with CM.

In various embodiments, a microbubble formulation may serve as a carrier to transfer THC and CBD to the systemic circulation via the intestinal lymphatic system following oral administration with lipids. Drugs that are transported via the intestinal lymphatic system can avoid hepatic first-pass metabolism and therefore achieve significantly higher bioavailability than after administration in lipid-free formulation. Thus, co-administration of microbubble lipids may substantially increase the systemic exposure to orally administered cannabis or cannabis-based medicines, and testing suggests that the primary mechanism of the increased absorption of cannabinoids in the presence of lipids may be intestinal lymphatic transport. The amount of lipids present in the microbubble formulation is sufficient to activate intestinal lymphatic transport and lead to increased systemic exposure to cannabinoids. The increase in systemic exposure to cannabinoids in humans is of potentially high clinical importance as it could turn a barely effective dose of orally administered cannabis into a highly effective one, or be a mechanism for adjustment of effective therapeutic dose.

OMB Formulation Delivery & Packaging

In various embodiments, the OMB formulations describe herein can be manufactured, stored and/or delivered in a variety of manners and packaging, including in disposal, single-use packaging. In at least one exemplary embodiment, an OMB formulation can be manufactured and packaged in airtight packaging, with the formulation capable or remaining in a stable and usable condition for an extended period of time, such as up to 2 years or longer. Desirably, the packaging will allow the OMB formulation to remain fully sealed until the time of ingestion and/or application, when the seal can be broken and the formulation ingested/applied quickly thereafter.

In at least one exemplary embodiment, such as for competitive sports applications, the OMB packaging could comprise a flexible or squeezable tube or other package, with a flip-top of other type cap forming an airtight closure on the tube. In use, the tube will desirably be grasped in a single hand, with the flip-top cap capable of being opened using the consumer's teeth, and the tube squeezed to expel some or all of the OMB formulation (i.e., some or all of the contents of the tube reservoir) into the open mouth of the consumer to be swallowed. In some embodiments the tube may be crushable so as to facilitate dispensing of more viscous and/or “thicker” OMB formulations, as well as to inhibit and/or prevent atmospheric air from being drawn back into the tube. In some embodiments the opening may closeable (i.e., a flip-top cap, for example) while other embodiments may be single-use only type openings.

In various embodiments, consumption of the OMB formulation may benefit from some level of mechanical and/or pneumatic delivery of the OMB formulation to the mouth, especially where the OMB formulation may not flow easily solely under the influence of gravity. For example, the OMB solution may be quite viscous, which may benefit from mechanical delivery assistance such as (1) a squeezable plastic bottle with a pressure sensitive seal, similar to sports bottles produced by various manufacturers (i.e., GATORADE water bottles used in various sports; (2) a syringe with a manual valve or pressure sensitive seal; (3) squeeze packs like an applesauce package in a “single serving” size; and/or (4) squeeze packs in larger volumes with a pressure sensitive seal. In other embodiment, standard beverage packaging such as aluminum cans and/or plastic water bottles may be advantageous for OMB formulation that are less viscous, flowable under gravity from the container, and/or may contain oxygen in lower concentrations (which may suitable depending on the desired application and/or field of use).

If desired, an OMB storage and delivery device could include multiple reservoirs for containing materials, including OMB formulations, which may allow for sequential consumption and/or allow for pre-mixing of contents prior to consumption and/or other introduction to an individual's anatomy. For example, it may be desirous to neutralize the acid within an athlete's stomach and/or digestive tract to facilitate the durability of the OMBs and/or the absorption of oxygen into the bloodstream. In such case, the OMB storage and delivery device could include a first reservoir containing an acid-neutralization liquid such as liquified calcium carbonate, and a second reservoir containing the OMB formulation, with the individual first consuming the acid neutralization liquid and then subsequently consuming the OMB formulation. In another embodiment, the reservoirs might be combinable prior to consumption, such as where the OMB formulation may be relatively viscous and an additional liquid (i.e., saline, electrolyte sports drink, or a flavored vitamin water or other liquid) can be added to slurrify the OMB formulation for consumption. This arrangement could allow the OMB formulation to remain relatively stable for transport, with mixing occurring immediately prior to consumption.

In various embodiments, the “consumption” of an OMB formulation could include situations where the OMB formulation in introduced “temporarily” into the digestive tract of the individual, and then is intentionally removed (via natural and/or artificial approaches) from the digestive tract prior to the occurrence of natural digestive responses. For example, an OMB formulation could be swallowed by an individual (and/or pumped into their stomach using a delivery system), and then the OMB formulation could be vomited by the user after a certain period of time (or could be pumped out of the stomach cavity at a desired time, etc.). In another exemplary embodiment the OMB formulation might comprise a mouthwash or oral swash formal, which could be held in the mouth of the user for a limited period of time and then spat out—possibly to be replaced with a fresh mouthful of the OMB formulation. In this manner, oxygen may be absorbed directly through mouth and/or throat tissues, and some amount of oxygen released by the OMBs may be “breathed in” by the individual as well. In another exemplary embodiment, the microbubbles may be impropriated into a nasal spray and/or flush delivery system.

In another exemplary embodiment, such as for medical applications, the OMB packaging could comprise a continuous OMB supply system, optionally with a refillable reservoir such as an IV drip bag-type packaging associated with an infusion pump or similar device. Such a system could include supply tubing and associated devices and an optional suction tube (for removal of used OMB products, if desired) and associated devices. Depending upon the desired application, the system could include additional access devices such as tubes for accessing the stomach or intestine through a naso-gastric or orogastric access, as well as placed percutaneously (i.e., long term G or J tubes). Other procedures could utilize enema and/or colonic tubes and/or related devices, including a supplemental oxygenation system that might include a supply tube inserted into a duodenum, a scavenging tube inserted into the colon or a distal ileum, and microbubbles from the supply device pumped into and through the entire length of the small bowel.

Microbubble Production

Oxygen microbubbles can be formulated with either a lipid monolayer shell, a biocompatible polymer shell, or a combination thereof. In addition to oxygen, the shell-stabilized microbubbles can be prepared with a variety of therapeutic gases. Additionally, these microbubbles can be formulated in a variety of biocompatible fluids that act as the continuous phase liquid for microbubble suspension. The lipids which may be used to prepare the gas and gaseous precursor filled microspheres used in the present invention include but are not limited to: lipids such as fatty acids, lysolipids, phosphatidylcholine with both saturated and unsaturated lipids including dioleoylphosphatidylcholine; dimyristoylphosphatidylcholine; dipentadecanoylphosphatidylcholine; dilauroylphosphatidylcholine; dipalmitoylphosphatidylcholine (DPPC); distearoylphosphatidylcholine (DSPC); phosphatidylethanolamines such as dioleoylphosphatidylethanolamine and dipalmitoylphosphatidylethanolamine (DPPE); phosphatidylserine; phosphatidylglycerol; phosphatidylinositol; sphingolipids such as sphingomyelin; glycolipids such as ganglioside GMI and GM2; glucolipids; sulfatides; glycosphingolipids; phosphatidic acids such as dipalymitoylphosphatidic acid (DPPA); pabnitic acid; stearic acid; arachidonic acid; oleic acid; lipids bearing polymers such as polyethyleneglycol, i.e., PEGylated lipids, chitin, hyaluronic acid or polyvinylpyrolidone; lipids bearing sulfonated mono-, di-, oligo- or polysaccharides; cholesterol, cholesterol sulfate and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of 6-8 carbons in length; synthetic phospholipids with asymmetric acyl chains (e.g., with one acyl chain of 6 carbons and another acyl chain of 12 carbons); ceramides; non-ionic liposomes including niosomes such as polyoxyethylene fatty acid esters, polyoxyethylene fatty alcohols, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene polymers, and polyoxyethylene fatty acid stearates; sterol aliphatic acid esters including cholesterol sulfate, cholesterol butyrate, cholesterol iso-butyrate, cholesterol palmitate, cholesterol stearate, lanosterol acetate, ergosterol palmitate, and phytosterol n-butyrate; sterol esters of sugar acids including cholesterol glucuroneide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl glucon-ate; esters of sugars and aliphatic acids including sucrose laurate, fructose laurate, sucrose palmitate, sucrose stearate, glucuronic acid, gluconic acid, accharic acid, and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; longchain alcohols including n-decyl alcohol, lauryl alcohol. myristyl alcohol, cetyl alcohol, and n-octadecyl alcohol; 6-(5-cholesten-3 yloxy)-1-thio- -D-galactopyranoside; digalactosyldiglyceride; 6-(5-cholesten-3-yloxy)hexyl-6-amino-6-deoxy-1-thio- -D-galactopyranoside; 6-(5-cholesten-3-yloxy)hexyl-6-amino-6-deoxyl-1-thio-a-D-mannopyranoside; 12-(((7′-diethylarninocoumarin-3-yl)carbonyl)methylamino)-octadecanoic acid; N-[12-(((7′-diethylaminocoumarin-3-yl)carbonyl)methyl-amino)octadecanoyl]-2-aminopalmiticacid; cholesteryl) 4′-trimethylammonio)butanoate; N-succinyldioleoylphosphatidylethanolamine; 1,2-dioleoyl-sn-glycerol; l,2-dipalmitoyl-sn-3-succinylglycerol; 1,3-dipalmitoyl-2-succinylglycerol; l-hexadecyl-2-palmitoyl glycerophosphoethanolamine and palmitoylhomocysteine, and/or combinations thereof.

If desired, a variety of cationic lipids such as DOTMA, N-[1-(2,3-dioleoyloxy)propyl]-N,N,N-trimethylammoium chloride; DITTAP, 1,2-dioleoyloxy-3-(trimethylammonio) propane; and DOTB, 1,2-dioleoyl-3-(4′-trimethyl-ammonio) butanoyl-sn-glycerol may be used. In general the molar ratio of cationic lipid to non-cationic lipid in the liposome may be, for example, 1:1000, 1:100, preferably, between 2:1 to 1:10, more preferably in the range between 1:1 to 1:2.5 and most preferably 1:1 (ratio of mole amount cationic lipid to mole amount non-cationic lipid, e.g., DPPC). A wide variety of lipids may comprise the non-cationic lipid when cationiclipid is used to construct the microsphere. Preferably, this non-cationic lipid is dipalmitoylphosphatidylcholine, dipalmitoylphosphatidylethanolamine or dioleoylphosphati-dylethanolamine. In lieu of cationic lipids as described above, lipids bearing cationic polymers such as polylysine or polyarginine, as well as alkyl phosphonates, alkyl phosphinates, and alkyl phosphites, may also be used to construct the microspheres.

In at least one exemplary embodiment, more preferred lipids can be phospholipids, preferably DPPC, DPPE, DPPA and DSPC, and most preferably DSPC.

In addition, examples of saturated and unsaturated fatty acids that may be used to prepare the stabilized micro- spheres used in the present invention, in the form of gas and gaseous precursor filled mixed micelles, may include molecules that may contain preferably between 12 carbon atoms and 22 carbon atoms in either linear or branched form. Hydrocarbon groups consisting of isoprenoid units and/or prenyl groups can be used as well. Examples of saturated fatty acids that are suitable include, but are not limited to, auric, myristic, palmitic, and stearic acids; examples of unsaturated fatty acids that may be used are, but are not limited to, lauroleic, physeteric, myristoleic, palmitoleic, petroselinic, and oleic acids; examples of branched fatty acids that may be used are, but are not limited to, isolauric, isomyristic, isopalmitic, and isostearic acids. In addition, to the saturated and unsaturated groups, gas and gaseous precursor filled mixed micelles can also be composed of 5 carbon isoprenoid and prenyl groups.

The biocompatible polymers useful as stabilizing compounds for preparing the gas and gaseous precursor filled microspheres used in the present invention can be of either natural, semi-synthetic or synthetic origin. As used herein, the term polymer denotes a compound comprised of two or more repeating monomeric units, and preferably 10 or more repeating monomeric units. The term semi-synthetic polymer, as employed herein, denotes a natural polymer that has been chemically modified in some fashion. Exemplary natural polymers suitable for use in the present invention include naturally occurring polysaccharides. Such polysaccharides include, for example, arabinans, fructans, fucans, galactans, galacturonans, glucans, mannans, xylans (such as, for example, inulin), levan, fucoidan, carrageenan, galatocarolose, pectic acid, pectin, amylose, pullulan, glycogen, amylopectin, cellulose, dextran, pustulan, chitin, agarose, keratan, chondroitan, dermatan, hyaluronic acid, alginic acid, xanthan gum, starch and various other natural homopolymer or heteropolymers such as those containing one or more of the following aldoses, ketoses, acids or amines: erythrose, threose, ribose, arabinose, xylose, lyxose, allose, altrose, glucose, mallose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Exemplary semi-synthetic polymers include carboxymethylcellulose, hydroxymethylcellulose, hydroxypropylmethylcellulose, methylcellulose, and methoxycellulose. Exemplary synthetic polymers suitable for use in the present invention include polyethylenes (such as, for example, polyethylene glycol, polyoxyethylene, and polyethylene terephthlate), polypropylenes (such as, for example, polypropylene glycol), polyurethanes (such as, for example, polyvinylalcohol (PVA), polyvinylchloride and polyvinylpyrrolidone), polyamides including nylon, polystyrene, polylactic acids, fluorinated hydrocarbons, fluorinated carbons (such as, for example, polytetrafluoroethylene), and polymethylmethacrylate, and derivatives thereof. Methods for the preparation of such polymer-based microspheres will be readily apparent to those skilled in the art, once armed with the present disclosure, when the present disclosure is coupled with information known in the art, such as that described and referred to in Unger, U.S. Pat No. 5,205,290, the disclosures of which are hereby incorporated herein by reference, in their entirety.

In one exemplary embodiment, oxygen microbubbles can be produced by mixing lipids at a 9:1 molar ratio of distearoyl phosphatidylcholine (DSPC) to poly(ethylene glycol)-40 stearate (PEG40S) in saline and sonicated at low power to create the small, unilamellar liposomes. O2 and liposomes (5 mg/mL) are then combined in the reaction chamber, where a high-power, ½-inch diameter, 20-kHz sonicator tip emulsifies the oxygen gas into micrometer-scale spheres around which phospholipid adsorbs from vesicles and micelles and self-assembles into a highly condensed (solid) monolayer coating. OMBs can be separated from macroscopic foam in a subsequent flotation container and collected in syringes and centrifuged (500 g for 3 min) to form concentrated OMBs. The sonication chamber and container are jacketed with circulating coolant to maintain a constant temperature of 20° C.

A desired OMB size distribution can be varied by choosing different residence times in the flotation container (e.g., 153 min for a 10-μm diameter cut-off; 38 min for a 20-μm diameter cut-off). Size distribution can be measured, for example, by electrical capacitance, light extinction/scattering, flow cytometry scatter, and optical microscopy. Alternatively, size selection may be unnecessary and may be removed from the process. OMB volume fraction is measured, for example, by gravimetric analysis and varied from 20-90 vol % by dilution with saline. Microbubble size and concentration is measured over time to investigate coalescence, Ostwald ripening and stability in storage.

The present disclosure also expressly incorporates by reference herein the disclosure of U.S. Pat. No. 8,481,077 entitled “Microbubbles and Methods for Oxygen Delivery” to Kheir et al, filed Feb. 22, 2012; U.S. Pat. No. 10,058,837 entitled “Systems, methods, and devices for production of gas-filled microbubbles” to Borden et al, filed Aug. 26, 2010; and U.S. Pat. No. 10,124,126 entitled “Systems and methods for ventilation through a body cavity” to Borden et al, filed Apr. 18, 2014. The entire disclosure of each of the publications, patent documents, and other references referred to herein is incorporated herein by reference in its entirety for all purposes to the same extent as if each individual source were individually denoted as being incorporated by reference.

Equivalents

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

General

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method of oxygenating the blood of an individual, comprising contacting a portion of an internal digestive tract of the individual with an aqueous formulation comprising microbubbles containing oxygen.

2. The method of claim 1, wherein the microbubbles are formulated from a lipid.

3. The method of claim 1, wherein the microbubbles are formulated from a polymer.

4. The method of claim 1, further comprising the step of ingesting the aqueous formulation.

5. The method of claim 4, wherein the aqueous formulation is self-administered by the individual.

6. The method of claim 4 wherein the aqueous formulation is administered by gastric feeding tube.

7. The method of claim 4 wherein the aqueous formulation is administered by nasogastric feeding tube.

8. The method of claim 4, wherein a majority of the microbubbles in the aqueous formulation comprise substantially-spherical gas-filled particles at or between 1 um to 1000 um in diameter in solution that are stabilized by an organic coating at the gas-liquid interface.

9. The method of claim 8, wherein the microbubbles are manufactured immediately prior to ingestion.

10. The method of claim 8, wherein the majority of the microbubbles in the aqueous formulation are between 1 to 100 um in diameter.

11. The method of claim 4, further comprising ingesting an additional compound that modifies at least a portion of the individual's digestive tract prior to ingesting the aqueous formulation.

12. The method of claim 4, wherein the aqueous formulation further comprises an additional compound that modifies a pH of at least a portion of the individual's digestive tract.

13. The method of claim 4, wherein the aqueous formulation further comprises at least one cannabinoid.

14. A beverage composition comprising water and at least 20% by volume of molecular oxygen, a majority of the molecular oxygen being encapsulated within substantially-spherical gas-filled particles between 1 um to 1000 um in diameter in solution that are stabilized by an organic coating at a gas-liquid interface.

15. The beverage composition of claim 14, further comprising a compound that modifies a pH of at least a portion of the individual's digestive tract.

16. The beverage composition of claim 14, further comprising a sweetening agent.

17. The beverage composition of claim 14, further comprising a cannabinoid.

18. The beverage composition of claim 14, further comprising an electrolyte sports drink.

19. The beverage composition of claim 14, further comprising a vitamin water drink.

20. The beverage composition of claim 14, further comprising nitric oxide.
